(19) United States
(12) Patent Application Publication
(10) Pub. No.: US 2023/0125108 A1
(43) Pub. Date: Apr. 27, 2023

(54) METHOD FOR DECOLORIZING ESSENTIAL OIL

(71) Applicant: TAKASAGO INTERNATIONAL CORPORATION, Tokyo (JP)

(72) Inventors: Hiroyuki MATSUDA, Kanagawa (JP); Naoto TERADA, Kanagawa (JP); Tadahiro HIRAMOTO, Kanagawa (JP)

(73) Assignee: TAKASAGO INTERNATIONAL CORPORATION, Tokyo (JP)

(21) Appl. No.: 17/911,747

(22) PCT Filed: Mar. 22, 2021
(86) PCT No.: PCT/JP2021/011798

§ 371 Date: Sep. 15, 2022

(30) Foreign Application Priority Data
Mar. 24, 2020 (JP) 2020-052999

(51) Int. Cl. C11B 9/02

(52) U.S. Cl. CPC C11B 9/02

(57) ABSTRACT

An object of the present invention is to provide a method for decolorizing an essential oil by which a change in a flavor of the essential oil is small even after decolorization. The first embodiment of the present invention relates to a method for decolorizing an essential oil, the method including an oxidation step of oxidizing an essential oil, in which the oxidation step includes a step of adding water and a phenol oxidase to the essential oil, and an addition amount of the water is 0.1 parts by mass to 1000 parts by mass with respect to 100 parts by mass of the essential oil.

### TECHNICAL FIELD

The present invention relates to a method for decolorizing an essential oil. The present invention also relates to a decolorized oil, a flavor or fragrance composition, a food or drink, an oral care product, or a fragrance or cosmetic.

### BACKGROUND ART

In the related art, from the viewpoint of improving the product value and the like, decolorization has been performed in a process of purifying oils such as essential oils. Various methods are used for decolorizing an oil, and for example, Patent Literature 1 discloses a technique of performing decolorization using activated carbon as an adsorbent.

### CITATION LIST

### Patent Literature

Patent Literature 1: Japanese Patent No. 4648199

### SUMMARY OF INVENTION

### Technical Problem

However, according to the studies of the present inventors, it has been found that, when the technique described in Patent Literature 1 is applied to an essential oil, a flavor of the essential oil may be largely changed after decolorization.

The present invention has been made in view of the above circumstances in the related art, and an object of the present invention is to provide a method for decolorizing an essential oil by which a change in a flavor of the essential oil is small even after decolorization.

### Solution to Problem

As a result of intensive studies to achieve the above object, the present inventors have found that the above problems can be solved by the following method, and have completed the present invention.
<1> A method for decolorizing an essential oil, the method including an oxidation step of oxidizing an essential oil,

in which the oxidation step includes a step of adding water and a phenol oxidase to the essential oil, and

an addition amount of the water is 0.1 parts by mass to 1000 parts by mass with respect to 100 parts by mass of the essential oil.
<2> The method for decolorizing the essential oil according to <1>, in which the phenol oxidase is at least one selected from the group consisting of laccase, peroxidase, and tyrosinase.
<3> The method for decolorizing the essential oil according to <1> or <2>, in which the phenol oxidase is laccase.
<4> A method for decolorizing an essential oil, the method including an oxidation step of oxidizing an essential oil,

in which the oxidation step includes a ventilation step of causing air or an oxygen gas to contact the essential oil under light shielding.
<5> The method for decolorizing the essential oil according to <4>, in which the ventilation step is performed under a condition of 100° C. or lower.
<6> The method for decolorizing the essential oil according to <4> or <5>, in which a ventilation volume of the air or the oxygen gas in the ventilation step is 0.01 L./min to 10 L/min per 1 L of the essential oil.
<7> The method for decolorizing the essential oil according to any one of <4> to <6>, further including a stirring step of stirring the essential oil.
<8> A decolorized oil obtained by the method for decolorizing the essential oil according to any one of <1> to <7>.
<9> A flavor or fragrance composition including the decolorized oil according to <8>.
<10> A food or drink, an oral care product, or a fragrance or cosmetic including the flavor or fragrance composition according to <9>.

### Advantageous Effects of Invention

An essential oil decolorized by a method for decolorizing an essential oil according to the present invention has just a small change in flavor compared to the essential oil before decolorization. In addition, the decolorization method of the essential oil according to the present invention can be easily performed.

### DESCRIPTION OF EMBODIMENTS

Hereinafter, the present invention will be described in detail, but these are examples of desirable embodiments, and the present invention is not limited to the contents thereof.

A method for decolorizing an essential oil according to the present invention includes an oxidation step of oxidizing an essential oil.

In a first embodiment of the present invention, the oxidation step includes a step of adding water and a phenol oxidase to an essential oil, and the addition amount of water is 0.1 to 1000 parts by mass with respect to 100 parts by mass of the essential oil.

It is considered that, when water and a phenol oxidase are added to the essential oil, pigment components are oxidized, and the essential oil can be decolorized. At this time, a change in a flavor of the essential oil is small even after decolorization.

In the present invention, the phrase “the change in the flavor of the essential oil is small” means that there is no significant change in a quality of aroma and the amount of an aroma component in the essential oil, and the entire flavor is well maintained.

When the addition amount of water is 0.1 parts by mass or more with respect to 100 parts by mass of the essential oil, the phenol oxidase can sufficiently come into contact with the essential oil. In contrast, in a case where the addition amount of water exceeds 1000 parts by mass with respect to 100 parts by mass of the essential oil, the change in the flavor of the essential oil after decolorization becomes large.

The addition amount of water is preferably 0.5 parts by mass or more and 200 parts by mass or less, and more preferably 1 part by mass or more and 50 parts by mass or less with respect to 100 parts by mass of the essential oil. The smaller addition amount of water is preferable since not only the change in flavor can be further reduced, but also a subsequent water removal step can be facilitated.

The addition amount of the phenol oxidase is preferably 0.001 parts by mass or more, more preferably 0.005 parts by mass or more, and still more preferably 0.01 parts by mass or more with respect to 100 parts by mass of water, from the viewpoint of obtaining a sufficient decolorization effect. Further, the addition amount of the phenol oxidase is preferably 20 parts by mass or less, more preferably 15 parts by mass or less, and still more preferably 12 parts by mass or less with respect to 100 parts by mass of water, from the viewpoint of solubility in water.

A method of adding water and the phenol oxidase to the essential oil is not particularly limited, and water and the phenol oxidase may be added at the same time, or may be added separately. A mixture obtained by adding water and a phenol oxidase to an essential oil is preferably subjected to a stirring treatment.

The stirring time may be, for example, 1 hour to 240 hours, and preferably 24 hours to 120 hours. At this time, the temperature of the mixture may be, for example, 15° C. to 55° C., preferably 30° C. to 45° C. The mixture may be allowed to stand under a condition of 15° C. to 55° C., for example, for 5 minutes to 240 minutes, and preferably for 30 minutes to 120 minutes after stirring.

As described above, the essential oil and the phenol oxidase can react with each other. In the present invention, it is preferable not to use a mediator and a coenzyme. When the mediator and the coenzyme are used, the activity of the phenol oxidase becomes too high, and the change in the flavor of the essential oil after decolorization may become large.

After the reaction, it is preferable that an aqueous layer is removed from the mixture, and an oil layer is heated to deactivate the phenol oxidase. A method for removing the aqueous layer is not particularly limited, and examples thereof include methods using separation with a separatory funnel, filtration such as natural filtration, vacuum filtration, pressure filtration, and centrifugal filtration, and a treatment with a dehydrating agent such as anhydrous magnesium sulfate, sodium sulfate, calcium carbonate, and molecular sieves. The heating temperature of the oil layer may be, for example, 80° C. to 100° C., and preferably 85° C. to 90° C., and the heating time may be, for example, 15 minutes to 60 minutes, and preferably 20 minutes to 40 minutes.

After the phenol oxidase is deactivated, it is preferable that the oil layer is dehydrated with a dehydrating agent, and the remaining oil layer is filtered. A method for filtering the oil layer is not particularly limited, and examples thereof include diatomaceous earth filtration, natural filtration, vacuum filtration, pressure filtration, centrifugal filtration, and the like.

As described above, the essential oil is decolorized to obtain the decolorized oil according to the present invention. The order of the above operations performed after the reaction may be reversed.

The essential oil used in the present invention may be a natural essential oil or a synthetic essential oil, and can be obtained by a commonly known method. Examples of the essential oil include citrus oils (grapefruit oils, orange oils, lemon oils, tangerine oils, lime oils, Yiizu oils, etc.), mint oils (peppermint oils, spearmint oils, Hakka oils, etc.), ginger oils, lavender oils, eucalyptus oils, rosemary oils, and the like.

The essential oil used in the present invention may be an essential oil obtained by concentrating a natural essential oil or a synthetic essential oil by means of distillation under reduced pressure, for example.

The water used in the present invention is not particularly limited, and examples thereof include ion-exchanged water, distilled water, ultrapure water, clean water, and the like.

The phenol oxidase used in the present invention may be commercially available or may be derived from microorganisms. In a case where a phenol oxidase derived from a microorganism is used, a culture solution of the microorganism that generates the phenol oxidase may be added to the essential oil. Examples of the microorganisms that generates a phenol oxidase include white-rot fungi, and specific examples thereof include Lentinula edodes, Trametes versicolor, and the like. In addition, examples of other microorganisms include Bacillus subtilis, Bacillus lichenifonnis, Pseudomonas extremorientalis, Pseudomonas aeruginosa, Pseudomonas putida, Streptomyces bikiniensis, Streptomyces cyanens, Azospirillum lipoferum, and the like.

Examples of the phenol oxidase include laccase, peroxidase, tyrosinase, and the like. Among these, laccase is preferable from the viewpoint of the decolorization effect.

In a second embodiment of the present invention, the oxidation step includes a ventilation step of allowing air or an oxygen gas to contact an essential oil under light shielding.

It is considered that, when the air or the oxygen gas contacts the essential oil under light shielding, pigment components are oxidized, and the essential oil can be decolorized. At this time, a change in a flavor of the essential oil is small even after decolorization.

From the viewpoint of obtaining the sufficient decolorization effect, the ventilation step is preferably performed under a condition of 100° C. or lower, more preferably performed under a condition of 0° C. to 100° C. and still more preferably performed under a condition of 20° C. to 80° C.

The ventilation volume of the air or the oxygen gas in the ventilation step is preferably 0.01 L/min to 10 L/min per 1 L of essential oil, more preferably 0.05 L/min to 5 L/rain per 1 L of essential oil, and still more preferably 0.1 L/min to 3 L/min per 1 L of essential oil, from the viewpoint of obtaining the sufficient decolorization effect.

In addition, it is preferable to stir the essential oil before the ventilation step, other the ventilation step, or during the ventilation step. The stirring time may be, for example, I hour to 1500 hours, and preferably 1 hour to 750 hours.

A flavor or fragrance composition according to the present invention contains the decolorized oil according to the present invention. The content of the decolorized oil in the flavor or fragrance composition according to the present invention is not strictly limited, and can be varied depending on the use of the flavor or fragrance composition. The content of the decolorized oil in the flavor or fragrance composition according to the present invention is preferably 0.001 mass % to 100 mass %, and more preferably 0.01 mass % to 100 mass %.

The flavor or fragrance composition of the present invention may contain a known flavor or fragrance component in addition to the decolorized

Examples of the known flavor or fragrance component include hydrocarbons such as α-pinene, β-pinene, limonene, p-cymene, and thujone; aliphatic alcohols such as octanol and p-tert-butylcyclohexanol; terpene-based alcohols such as menthol, citronella and geraniol; aromatic alcohols such as benzyl alcohol and phenylethyl alcohol; aliphatic aldehydes; terpene-based aldehydes; aromatic aldehydes; acetals; chain ketones; cyclic ketones such as damascones, β-ionone, and methylionone; terpene-based ketones such as carvone, menthone, isomenthone, and camphor; aromatic ketones such as acetophenone and raspberry ketone; ethers such as dibenzyl ether; oxides such as linalool oxide and rose oxide;

musks such as cycropentadecanolide and cyclohexadecanolide; lactones such as γ-nonalactone, γ-undecalactone, and coumarin; aliphatic esters such as acetate esters and propionate esters; and aromatic esters such as benzoic acid esters and phenyl acetic acid esters.

The flavor or fragrance composition according to the present invention may further contain ethanol, isopropyl alcohol, ethylene glycol, propylene glycol, dipropylene glycol, butylene, glycol, pentylene glycol, hexylene glycol, polyethylene glycol, diethyl phthalate, isopropyl myristate, triethyl citrate, benzyl benzoate, glycerin, triacetin, benzyl alcohol, paraffin, isoparaffin, rosin ester derivatives such as Hercolyn, glycol ethers such as 3-methoxy-3-methyl-1-butanol, ethyl carbitol (diethylene glycol monoethyl ether), ethylene glycol monomethyl ether, propylene glycol monomethyl ether, ethylene glycol monoethyl ether, dipropylene glycol monomethyl ether, tripropylene glycol methyl ether, dipropylene glycol dimethyl ether, dipropylene glycol propyl ether, dipropylene glycol methyl ether acetate, and dipropylene glycol butyl ether, terpene resins such as pinene polymers, silicones such as cyclic silicones, solvents such as water, and fixatives.

The flavor or fragrance composition according to the present invention may further contain known components such as a higher alcohol, a surfactant, an antioxidant, an ultraviolet absorber, a chelating agent, a solubilizing agent, a stabilizer, a cooling agent, a preservative, an antibacterial agent, a bactericidal agent, an antifungal agent, an insecticidal component, and a pigment, if necessary.

The flavor or fragrance composition according to the present invention may be obtained, for example, by mixing and stirring components, and heating or the like may be performed as desired. The content of the components may be appropriately adjusted.

A food or drink, an oral care product, or a fragrance or cosmetic according to the present invention contains the flavor or fragrance composition according to the present invention. The content of the flavor or fragrance composition in the food or drink, the oral care product, or the fragrance or cosmetic according to the present invention is not strictly limited, and can be varied depending on the use of the food or drink, the oral care product, or the fragrance or cosmetic. The content of the flavor or fragrance composition in the food or drink, the oral care product, or the fragrance or cosmetic according to the present invention is preferably 0.0001 mass % to 10 mass %, and more preferably 0.001 mass % to 1 mass %.

The form of the food or drink, the oral care product, or the fragrance or cosmetic according to the present invention is not limited, and may be any of a liquid, a solid, a semi-solid, and a fluid.

Examples of the food or drink include: liquid products such as fruit beverages, vegetable beverages, carbonated beverages, sports drink, coffee beverages, tea, black tea, yogurt beverages, lactic acid bacteria beverages, nutritional drinks, soup, and mentsuyu; solid products such as candy, gum, gummies, jellies, chocolates, ice creams, hams, sausages, and snacks; and fluid products such as curries, stew, rice with hashed meat, sauces, tare sauces, dressings, and heavy creams.

Examples of the oral care products include toothpastes, dentifrices, liquid toothpastes, mouthwashes, gingival massage creams, local coating agent, troches, chewing gums, and the like.

Examples of the fragrance or cosmetic include fragrance products (perfume, eau de parfum, eau de toilette, eau de cologne, etc.), foundation cosmetics (facial wash creams, vanishing creams, cleansing creams, cold creams, massage creams, milky lotions, skin lotions, beauty lotions, facial packs, makeup removers, etc.), finishing cosmetics (foundations, face powders, solid face powders, talcum powders, rouges, lip balms, cheek rouges, eye liners, mascara, eye shadows, eyebrow pencils, eye packs, nail enamels, enamel removers, etc.), hair cosmetics (pomade, brilliantine, hair set lotions, hair sticks, hair solids, hair oils, hair treatments, hair creams, hair tonics, hair liquids, hair sprays, bandolines, revitalizing hair tonics, hair dyes, etc.), suntan cosmetics (suntan products, sun-screen products, etc.), medicated cosmetics (antiperspirants, after-shaving lotions, after-shaving gels, permanent wave agents, medicated soaps, medicated shampoos, medicated skin cosmetics, etc.), and the like.

### EXAMPLES

Hereinafter, the present invention will be described in detail with reference to Examples, but the present invention is not limited thereto.

### Test Example 1

### Example 1-1

To 17 of a five-fold concentrated product of a grapefruit oil having a Gardner color number of 18, 3 g of ion-exchanged water and 0.34 g of “laccase Y-120” (manufactured by Ammo Enzyme Inc.) were added, and the mixture was subjected to a stirring treatment at 45° C. for 48 hours and then allowed to stand at 25° C. for 30 minutes.

In the present specification, the five-fold concentrated product of the grapefruit oil means a product obtained by removing unnecessary terpenes from the grapefruit oil and increasing the content of useful oxygen-containing compounds by five times. The five-fold concentrated product of the grapefruit oil can be obtained by heating and distilling the grapefruit oil under reduced pressure.

After the standing, an aqueous layer was removed with a separatory funnel, and an oil layer was heated at 85° C. for 30 minutes to deactivate the enzyme. After deactivation of the enzyme, the oil layer was filtered with filter paper to obtain 14.5 g of a five-fold concentrated product of a grapefruit oil.

### Comparative Example 1-1

A column was filled with 160 g of “activated carbon GAC1240” (manufactured by CABOT NORIT), and 1000 g of a grapefruit oil having a Gardner color number of 15 was fed thereto and subjected to a circulation treatment at 25° C. for 6 hours. After the circulation treatment, the grapefruit oil was filtered with filter paper, followed by performing a distillation treatment, and 190 g of a five-fold concentrated product of the grapefruit oil was obtained.

### Analysis of Oil

The five-fold concentrated product of the grapefruit oil obtained in Example 1-1 and the five-fold concentrated product of the grapefruit oil obtained in Comparative Example 1-1, and an untreated five-fold concentrated product of a grapefruit oil were measured in terms of the Gardner color number, the specific gravity, the refractive index, and the flavor component concentration (concentrations of nootkatone and auraptene). The results are shown in Table 1.

The Gardner color number was measured by comparing grapefruit oil with a Gardner sample. When the color of the oil is between colors of the two Gardner samples, the Gardner color number of the Gardner sample closest to that of the oil is determined, and “+” is added when the color is darker than the number of colors, and “−” is added when the color is lighter than the color number. The specific gravity was measured with a specific gravity meter (“DMA 4500M” manufactured by Anton Paar). The refractive index was measured with a refractometer (“RX-5000i” manufactured by ATAGO). The flavor component concentration was quantified by GC-FID after each flavor component peak was identified by GC-MS.

### Sensory Evaluation

The five-fold concentrated product of the grapefruit oil obtained in Example 1-1 and the five-fold concentrated product of the grapefruit oil obtained in Comparative Example 1-1 were subjected to sensory evaluation by five expert panelists as follows. First, ethanol solutions containing 5 mass % of the five-fold concentrated product of grapefruit oil were prepared. Each of the solutions was added to water in an amount of 0.1 mass % to perfume the water, and an untreated product was evaluated as a control based on the following criteria. The average scores, comments, and comprehensive evaluations of five panelists are shown in Table 1. When the average score was 4 or more, the sensory evaluation was determined to be acceptable.
5: The aroma was equivalent to that of the control.
4: The aroma was very slightly different from that of the control.
3: The aroma was slightly different from that of the control.
2: The aroma was greatly different from that of the control.
1: The aroma was completely different from that of the control.

TABLE 1

| | Addition amount | | | | | | | | | |
|---|---|---|---|---|---|---|---|---|---|---|
| | (part(s) by mass) | | | | | | | | | |
| | Water (with | Enzyme (with | | | | | | | | |
| | respect to | respect to | | | | | | Sensory evaluation | | |
| | 100 parts | 100 parts | Gardner | | | Flavor component | | Evaluation | | |
| | by mass of | by mass of | color | Specific | Refractive | concentration (mass %) | | score | | Comprehensive |
| | essential oil) | water) | number | gravity | index | Nootkatone | Auraptene | (average) | Comment | evaluation |
| Untreated | — | — | 18 | 0.906 | 1.493 | 0.24 | 4.83 | — | — | — |
| product | | | | | | | | | | |
| Example 1-1 | 17.6 | 11.3 | 12+ | 0.906 | 1.493 | 0.23 | 4.94 | 4.5 | Equivalent | No flavor change, |
| | | | | | | | | | to control, | can decolorize |
| | | | | | | | | | cannot | efficiently and |
| | | | | | | | | | distinguish | significantly |
| | | | | | | | | | sensorilly | |
| Comparative | — | — | 12+ | 0.867 | 1.478 | 0.17 | 0.55 | 1.5 | volume | Although can |
| Example 1-1 | | | | | | | | | feeling is | decolorize |
| | | | | | | | | | not sufficient, | efficiently and |
| | | | | | | | | | not equivalent | significantly, |
| | | | | | | | | | to control, can | flavor change |
| | | | | | | | | | distinguish | was large |
| | | | | | | | | | sensorilly | |

From the results in Table 1. it was found that in Example 1-1, the grapefruit oil was decolorized without substantially changing the specific gravity, the refractive index, and the flavor component concentration as compared with the untreated product. In addition, in the sensory evaluation, Example 1-1 was equivalent to the untreated product.

### Test Example 2

### Example 2-1

To 17 g of a five-fold concentrated product of a grapefruit oil having a Gardner color number of 15, 3 g of ion-exchanged water and 0.085 g of “laccase Y-120” (manufactured by Amano Enzyme Inc.) were added, and the mixture was subjected to a stirring treatment at 30° C. for 24 hours and then allowed to stand at 25° C. for 30 minutes.

After the standing, 14.1 g of a five-fold concentrated product of a grapefruit oil was obtained in the same manner as in Example 1-1.

### Example 2-2

A five-fold concentrated product (13.9 g) of a grapefruit oil was obtained in the same manner as in Example 2-1 except that 0.085 g of “peroxidase” (manufactured by FUJIFILM Wako Pure Chemical Corporation) was used instead of 0.085 g of “laccase Y-120”.

The grapefruit oils obtained in Examples 2-1 and 2-2 and the untreated grapefruit oils were measured in terms of the Gardner color number, the specific gravity, the refractive index, and the flavor component concentration (concentrations of nootkatone and auraptene) in the same manner as in Test Example 1. In addition, sensory evaluation was performed in the same manner as in Test Example 1. The results are shown in Table 2.

TABLE 2

| | Addition amount | | | | | | | | | |
|---|---|---|---|---|---|---|---|---|---|---|
| | (part(s) by mass) | | | | | | | | | |
| | Water (with | Enzyme (with | | | | | | | | |
| | respect to | respect to | | | | | | Sensory evaluation | | |
| | 100 parts | 100 parts | Gardner | | | Flavor component | | Evaluation | | |
| | by mass of | by mass of | color | Specific | Refractive | concentration (mass %) | | score | | Comprehensive |
| | essential oil) | water) | number | gravity | index | Nootkatone | Auraptene | (average) | Comment | evaluation |
| Untreated | — | — | 15 | 0.909 | 1.494 | 0.22 | 4.66 | — | — | — |
| product | | | | | | | | | | |
| Example 2-1 | 17.6 | 2.8 | 12 | 0.909 | 1.494 | 0.21 | 4.69 | 4.4 | Equivalent | No flavor change, |
| | | | | | | | | | to control, | can decolorize |
| | | | | | | | | | cannot | efficiently and |
| | | | | | | | | | distinguish | significantly |
| | | | | | | | | | sensorilly | |
| Example 2-2 | 17.6 | 2.8 | 11 | 0.909 | 1.494 | 0.22 | 4.67 | 4.3 | Equivalent | No flavor change, |
| | | | | | | | | | to control, | can decolorize |
| | | | | | | | | | cannot | efficiently and |
| | | | | | | | | | distinguish | significantly |
| | | | | | | | | | sensorilly | |

From the results of Table 2, it was found that in Examples 2-1 and 2-2, the grapefruit oils were decolorized without substantially changing the specific gravity, the refractive index, and the flavor component concentration as compared with the untreated product, In addition, in the sensory evaluation, Examples 2-1 and 2-2 were equivalent to the untreated product.

### Test Example 3

### Example 3-1

To 17 g of an orange oil having a Gardner color number of 9, 3 g of ion-exchanged water and 0.34 g of “laccase Y-120” (manufactured by Amano Enzyme Inc.) were added, and the mixture was subjected to a stirring treatment at 45° C. for 24 hours and then allowed to stand at 25° C. for 30 minutes.

After the standing, 13.4 g of an orange oil was obtained in the same manner as in Example 1-1.

The orange oil obtained in Example 3-1 and the untreated orange oil were measured in terms of the Gardner color number in the same manner as in Test Example 1. In addition, sensory evaluation was performed in the same manner as in Test Example 1. The results are shown in Table 3.

TABLE 3

| | Addition amount | | | | | |
|---|---|---|---|---|---|---|
| | (part(s) by mass) | | | | | |
| | Water (with | Enzyme (with | | | | |
| | respect to | respect to | | Sensory evaluation | | |
| | 100 parts | 100 parts | Gardner | Evaluation | | |
| | by mass of | by mass of | color | score | | Comprehensive |
| | essential oil) | water) | number | (average) | Comment | evaluation |
| Untreated | — | — | 9 | — | — | — |
| product | | | | | | |
| Example 3-1 | 17.6 | 11.3 | 6 | 4.2 | Equivalent | No flavor change, |
| | | | | | to control, | can decolorize |
| | | | | | cannot | efficiently and |
| | | | | | distinguish | significantly |
| | | | | | sensorilly | |

From the results in Table 3, it was found that the orange oil was decolorized in Example 3-1 as compared with the untreated product. In addition, in the sensory evaluation, Example 3-1 was equivalent to the untreated product.

### Test Example 4

### Example 4-1

To 17 g of a grapefruit oil having a Gardner color number of 15, 3 g of ion-exchanged water and 0.17 g of “laccase Y-120” (manufactured by Amano Enzyme Inc.) were added, and the mixture was subjected to a stirring treatment at 45° C. for 24 hours and then allowed to stand at 25° C. for 30 minutes.

After the standing, 13.6 g of a grapefruit oil was obtained in the same manner as in Example 1-1.

The grapefruit oil obtained in Example 4-1 and the untreated grapefruit oil were measured in terms of the Gardner color number in the same manner as in Test Example 1. In addition, sensory evaluation was performed in the same manner as in Test Example 1. The results are shown in Table 4.

TABLE 4

| | Addition amount | | | | | |
|---|---|---|---|---|---|---|
| | (part(s) by mass) | | | | | |
| | Water (with | Enzyme (with | | | | |
| | respect to | respect to | | Sensory evaluation | | |
| | 100 parts | 100 parts | Gardner | Evaluation | | |
| | by mass of | by mass of | color | score | | Comprehensive |
| | essential oil) | water) | number | (average) | Comment | evaluation |
| Untreated | — | — | 15 | — | — | — |
| product | | | | | | |
| Example 4-1 | 17.6 | 5.7 | 12 | 4.2 | Equivalent | No flavor change, |
| | | | | | to control, | can decolorize |
| | | | | | cannot | efficiently and |
| | | | | | distinguish | significantly |
| | | | | | sensorilly | |

From the results in Table 4, it was found that the grapefruit oil was decolorized in Example 4-1 as compared with the untreated product. In addition, in the sensory evaluation, Example 4-1 was equivalent to the untreated product.

### Test Example 5

### Example 5-1

To 291.7 g of a. grapefruit oil having a Gardner color number of 14+, 58.3 g of ion-exchanged water and 2.935 g of “laccase Y-120” (manufactured by Amano Enzyme Inc.) were added, and the mixture was subjected to a stirring treatment at 45° C. for 24 hours and then allowed to stand at 25° C. for 30 minutes.

After the standing, 273.7 g of a grapefruit oil was obtained in the same manner as in Example 1-1.

### Example 5-2

A grapefruit oil (156.3 g) was obtained in the same manner as in Example 5-1 except that the amount of the grapefruit oil used was changed to 175 g, and the addition amount of the ion-exchanged water was changed to 175 g.

### Example 5-3

A grapefruit oil (42.5 g) was obtained in the same manner as in Example 5-1 except that the amount of the grapefruit oil used was changed to 50 g and the addition amount of the ion-exchanged water was changed to 300 g.

### Example 5-4

A grapefruit oil (28.3 g) was obtained in the same manner as in Example 5-1 except that the amount of the grapefruit oil used was changed to 35 g and the addition amount of the ion-exchanged water was changed to 315 g.

### Comparative Example 5-1

A grapefruit oil (11.5 g) was obtained in the same manner as in Example 5-1 except that the amount of the grapefruit oil used was changed to 14 g and the addition amount of the ion-exchanged water was changed to 336 g.

The grapefruit oils obtained in Examples 5-1 to 5-4 and Comparative Example 5-1 and the untreated grapefruit oils were measured in terms of the Gardner color number in the same manner as in Test Example 1. In addition, sensory evaluation was performed in the same manner as in Test Example 1. The results are shown in Table 5.

TABLE 5

| | Addition amount | | | | | |
|---|---|---|---|---|---|---|
| | (part(s) by mass) | | | | | |
| | Water (with | Enzyme (with | | | | |
| | respect to | respect to | | Sensory evaluation | | |
| | 100 parts | 100 parts | Gardner | Evaluation | | |
| | by mass of | by mass of | color | score | | Comprehensive |
| | essential oil) | water) | number | (average) | Comment | evaluation |
| Untreated | — | — | 14+ | — | — | — |
| product | | | | | | |
| Example 5-1 | 20 | 5.0 | 11 | 4.4 | Equivalent | No flavor change, |
| | | | | | to control, | can decolorize |
| | | | | | cannot | efficiently and |
| | | | | | distinguish | significantly |
| | | | | | sensorilly | |
| Example 5-2 | 100 | 1.7 | 10 | 4.3 | Equivalent | No flavor change, |
| | | | | | to control, | can decolorize |
| | | | | | cannot | efficiently and |
| | | | | | distinguish | significantly |
| | | | | | sensorilly | |
| Example 5-3 | 600 | 0.98 | 8 | 4.2 | Equivalent, | No flavor change, |
| | | | | | to control, | can decolorize |
| | | | | | cannot | efficiently and |
| | | | | | distinguish | significantly |
| | | | | | sensorilly | |
| Example 5-4 | 900 | 0.93 | 8 | 4.1 | Equivalent | No flavor change, |
| | | | | | to control, | can decolorize |
| | | | | | cannot | efficiently and |
| | | | | | distinguish | significantly |
| | | | | | sensorilly | |
| Comparative | 2400 | 0.87 | 7 | 1.7 | Weak volume | Although can |
| Example 5-1 | | | | | feeling, large | decolorize |
| | | | | | difference in | efficiently and |
| | | | | | aroma note, | significantly, |
| | | | | | not equivalent | flavor change |
| | | | | | to control, | was large |
| | | | | | can distinguish | |
| | | | | | sensorilly | |

From the results of Table 5, it was found that the grapefruit oils were decolorized in Examples 5-1 to 5-4 as compared with the untreated product. In addition, in the sensory evaluation, Examples 5-1 to 5-4 were equivalent to the untreated product.

### Test Example 6

### Example 6-1

To 1089 g of a grapefruit oil having a Gardner color number of 15, 11 g of ion-exchanged water and 0.0011 g of “laccase Y-120” (manufactured by Amano Enzyme Inc.) were added, and the mixture was subjected to a stirring treatment at 45° C. for 24 hours and then allowed to stand at 25° C. for 30 minutes.

After the standing, 1001.9 g of a grapefruit oil was obtained in the same manner as in Example 1-1.

### Example 6-2

A grapefruit oil (999.3 g) was obtained in the same manner as in Example 6-1 except that the addition amount of “laccase Y-120” was changed to 0.11 g.

### Example 6-3

A grapefruit oil (997.1 g) was obtained in the same manner as in Example 6-1 except that the addition amount of “laccase Y-120” was changed to 1.1 g.

### Example 6-4

To 1000 g of a grapefruit oil haying a Gardner color number of 15, 100 g of ion-exchanged water and 001 g of “laccase Y-120” (manufactured by Amano Enzyme Inc.) were added, and the mixture was subjected to a stirring treatment at 45° C. for 24 hours and then allowed to stand at 25° C. for 30 minutes.

After the standing, 951.2 g of a grapefruit oil was obtained in the same manner as in Example 1-1.

### Example 6-5

A grapefruit oil (949 g) was obtained in the same manner as in Example 6-4 except that the addition amount of “laccase Y-120” was changed to 1 g,

### Example 6-6

A grapefruit oil (948,5 g) was obtained in the same manner as in Example 6-4 except that the addition amount of “laccase Y-120” was changed to 10 g.

### Example 6-7

To 550 g of a grapefruit oil having a Gardner color number of 15, 550 g of ion-exchanged water and 0.055 g of “laccase Y-120” (manufactured by Amano Enzyme Inc.) were added, and the mixture was subjected to a stirring treatment at 45° C. liar 24 hours and then allowed to stand at 25° C. for 30 minutes.

After the standing, 511.5 g of a grapefruit oil was obtained in the same manner as in Example 1-1.

### Example 6-8

A grapefruit oil (510 g) was obtained in the same manner as in Example 6-7 except that the addition amount of laccase Y-120″ was changed to 5.5 g.

### Example 6-9

A grapefruit oil (508.2 g) was obtained in the same manner as in Example 6-7 except that the addition amount of “laccase Y-120” was changed to 55 g.

### Example 6-10

To 100 g of a grapefruit oil having a Gardner color number of 15, 1000 g of ion exchanged water and 0.1 g of “laccase Y-120” (manufactured by Amano Enzyme Inc.) were added, and the mixture was subjected to a stirring treatment at 45° C. for 24 hours and then allowed to stand at 25° C. for 30 minutes.

After the standing, 88.4 g of grapefruit oil was obtained in the same manner as in Example 1-1.

### Example 6-11

A grapefruit oil (87.5 g) was obtained in the same manner as in Example 6-10 except that the addition amount of “laccase Y-120” was changed to 10 g.

### Example 6-12

A grapefruit oil (87 g) was obtained in the same manner as in Example 6-10 except that the addition amount of laccase Y-120″ was changed to 100 g.

### Comparative Example 6-1

To 50 g of a grapefruit oil having a Gardner color number of 15, 1050 g of ion-exchanged water and 0.105 g of “laccase Y-120” (manufactured by Ammo Enzyme Inc.) were added, and the mixture was subjected to a stirring treatment at 45° C. for 24 hours and then allowed to stand at 25° C. for 30 minutes.

After the standing, 42.6 g of a grapefruit oil was obtained in the same manner as in Example 1-1.

### Comparative Example 6-2

A grapefruit oil (41.5 g) was obtained in the same manner as in Comparative Example 6-1 except that the addition amount of “laccase Y-120” was changed to 10.5 g.

### Comparative Examples 6-3

A grapefruit oil (40.1 g) was obtained in the same manner as in Comparative Example 6-1 except that the addition amount of “laccase Y-120” was changed to 10.5 g.

The grapefruit oils obtained in Examples 6-1 to 6-12 and Comparative Examples 6-1 to 6-3 and the untreated grapefruit oil were measured in terms of the Gardner color number in the same manner as in Test Example 1. In addition, sensory evaluation was performed in the same manner as in Test Example 1. The results are shown in Tables 6 and 7.

TABLE 6

| | Addition amount | | | | | |
|---|---|---|---|---|---|---|
| | (part(s) by mass) | | | | | |
| | Water (with | Enzyme (with | | | | |
| | respect to | respect to | | Sensory evaluation | | |
| | 100 parts | 100 parts | Gardner | Evaluation | | |
| | by mass of | by mass of | color | score | | Comprehensive |
| | essential oil) | water) | number | (average) | Comment | evaluation |
| Untreated | — | — | 15 | — | — | — |
| product | | | | | | |
| Example 6-1 | 1 | 0.01 | 14+ | 4.5 | Equivalent | No flavor change, |
| | | | | | to control, | can decolorize |
| | | | | | cannot | efficiently and |
| | | | | | distinguish | significantly |
| | | | | | sensorilly | |
| Example 6-2 | 1 | 1 | 14 | 4.5 | Equivalent | No flavor change, |
| | | | | | to control, | can decolorize |
| | | | | | cannot | efficiently and |
| | | | | | distinguish | significantly |
| | | | | | sensorilly | |
| Example 6-3 | 1 | 10 | 13+ | 4.4 | Equivalent | No flavor change, |
| | | | | | to control, | can decolorize |
| | | | | | cannot | efficiently and |
| | | | | | distinguish | significantly |
| | | | | | sensorilly | |
| Example 6-4 | 10 | 0.01 | 14 | 4.5 | Equivalent | No flavor change, |
| | | | | | to control, | can decolorize |
| | | | | | cannot | efficiently and |
| | | | | | distinguish | significantly |
| | | | | | sensorilly | |
| Example 6-5 | 10 | 1 | 13 | 4.4 | Equivalent | No flavor change, |
| | | | | | to control, | can decolorize |
| | | | | | cannot | efficiently and |
| | | | | | distinguish | significantly |
| | | | | | sensorilly | |
| Example 6-6 | 10 | 10 | 12 | 4.3 | Equivalent | No flavor change, |
| | | | | | to control, | can decolorize |
| | | | | | cannot | efficiently and |
| | | | | | distinguish | significantly |
| | | | | | sensorilly | |
| Example 6-7 | 100 | 0.01 | 13 | 4.4 | Equivalent | No flavor change, |
| | | | | | to control, | can decolorize |
| | | | | | cannot | efficiently and |
| | | | | | distinguish | significantly |
| | | | | | sensorilly | |
| Example 6-8 | 100 | 1 | 11 | 4.3 | Equivalent | No flavor change, |
| | | | | | to control, | can decolorize |
| | | | | | cannot | efficiently and |
| | | | | | distinguish | significantly |
| | | | | | sensorilly | |

TABLE 7

| | Addition amount | | | | | |
|---|---|---|---|---|---|---|
| | (part(s) by mass) | | | | | |
| | Water (with | Enzyme (with | | | | |
| | respect to | respect to | | Sensory evaluation | | |
| | 100 parts | 100 parts | Gardner | Evaluation | | |
| | by mass of | by mass of | color | score | | Comprehensive |
| | essential oil) | water) | number | (average) | Comment | evaluation |
| Example 6-9 | 100 | 10 | 10 | 4.2 | Equivalent | No flavor change, |
| | | | | | to control, | can decolorize |
| | | | | | cannot | efficiently and |
| | | | | | distinguish | significantly |
| | | | | | sensorilly | |
| Example 6-10 | 1000 | 0.01 | 10 | 4.2 | Equivalent | No flavor change, |
| | | | | | to control, | can decolorize |
| | | | | | cannot | efficiently and |
| | | | | | distinguish | significantly |
| | | | | | sensorilly | |
| Example 6-11 | 1000 | 1 | 8 | 4.2 | Equivalent | No flavor change, |
| | | | | | to control, | can decolorize |
| | | | | | cannot | efficiently and |
| | | | | | distinguish | significantly |
| | | | | | sensorilly | |
| Example 6-12 | 1000 | 10 | 7 | 4.1 | Equivalent | No flavor change, |
| | | | | | to control, | can decolorize |
| | | | | | cannot | efficiently and |
| | | | | | distinguish | significantly |
| | | | | | sensorilly | |
| Comparative | 2100 | 0.01 | 9 | 1.9 | Weak volume | Although can |
| Example 6-1 | | | | | feeling, large | decolorize |
| | | | | | difference in | efficiently and |
| | | | | | aroma note, not | significantly, |
| | | | | | equivalent to | flavor change |
| | | | | | control, can | was large |
| | | | | | distinguish | |
| | | | | | sensorilly | |
| Comparative | 2100 | 1 | 7+ | 1.8 | Weak volume | Although can |
| Example 6-2 | | | | | feeling, large | decolorize |
| | | | | | difference in | efficiently and |
| | | | | | aroma note, not | significantly, |
| | | | | | equivalent to | flavor change |
| | | | | | control, can | was large |
| | | | | | distinguish | |
| | | | | | sensorilly | |
| Comparative | 2100 | 10 | 6+ | 1.8 | Weak volume | Although can |
| Example 6-3 | | | | | feeling, large | decolorize |
| | | | | | difference in | efficiently and |
| | | | | | aroma note, not | significantly, |
| | | | | | equivalent to | flavor change |
| | | | | | control, can | was large |
| | | | | | distinguish | |
| | | | | | sensorilly | |

From the results of Tables 6 and 7, it was found that the grapefruit oils were decolorized in Examples 6-1 to 6-12 as compared with the untreated product. In addition, in the sensory evaluation, Examples 6-1 to 6-12 were equivalent to the untreated product.

### Test Example 7

### Example 7-1

A grapefruit oil was obtained by stirring 1000 g of a grapefruit oil having a Gardner color number of 14—for 72 hours at 37° C. and a ventilation volume of 0.01 vvm under light shielding.

In the present specification, the ventilation volume vvm means the ratio of the volume of air or oxygen gas which contacts the oil to the volume of the oil per minute. In Test Example 7, air was used for the ventilation, and the ventilation and the stirring treatment were performed at the same time.

### Example 7-2

A grapefruit oil was obtained by stirring 1000 g of a grapefruit oil having a Gardner color number of 14—for 72 hours at 37° C. and a ventilation volume of 0.1 vvm under light shielding.

### Example 7-3

A grapefruit oil was obtained by stirring 1000 g of a grapefruit oil having a Gardner color number of 14—for 72 hours at 37° C. and a ventilation volume of 1 vvm under light shielding.

### Example 7-4

A grapefruit oil was obtained by stirring 1000 g of a grapefruit oil having a Gardner color number of 14—for 72 hours at 37° C. and a ventilation volume of 2 vvm under light shielding.

### Example 7-5

A grapefruit oil was obtained by stirring 1000 g of a grapefruit oil having a Gardner color number of 14—for 72 hours at 37° C. and a ventilation volume of 5 vvm under light shielding.

### Example 7-6

A grapefruit oil was obtained by stirring 1000 g of a grapefruit oil having a Gardner color number of 14—for 72 hours at 37° C. and a ventilation volume of 10 vvm under light shielding.

### Example 7-7

A grapefruit oil was obtained by stirring 1000 g of a grapefruit oil having a Gardner color number of 14—for 144 hours at 37° C. and a ventilation volume of 0.1 vvm under light shielding.

### Example 7-8

A grapefruit oil was obtained by stirring 1000 g of a grapefruit oil having a Gardner color number of 14—for 24 hours at 50° C. and a ventilation volume of 0.1 vvm under light shielding.

### Example 7-9

A grapefruit oil was obtained by stirring 1000 g of a grapefruit oil having a Gardner color number of 14—for 240 hours at 25° C. and a ventilation volume of 0.1 vvm under light shielding.

### Example 7-10

A grapefruit oil was obtained by stirring 1000 g of a grapefruit oil having a Gardner color number of 14—for 720 hours at 10° C. and a ventilation volume of 0.1 vvm under light shielding.

### Example 7-11

A grapefruit oil was obtained by stirring 1000 g of a grapefruit oil having a Gardner color number of 14—for 10 hours at 75° C. and a ventilation volume of 0.1 vvm under light shielding.

### Example 7-12

A grapefruit oil was obtained by stirring 1000 g of a grapefruit oil having a Gardner color number of 14—for 5 hours at 100° C. and a ventilation volume of 0.1 vvm under light shielding.

### Comparative Example 7-1

A grapefruit oil was obtained by stirring 1000 g of a grapefruit oil having a Gardner color number of 14—for 72 hours at 37° C. under light shielding after the air in a vessel was replaced with a nitrogen gas and then the vessel was sealed.

### Comparative Example 7-2

A grapefruit oil was obtained by stirring 1000 g of a grapefruit oil having a Gardner color number of 14—for 72 hours at 37° C. and a ventilation volume of 15 vvm under light shielding.

### Comparative Example 7-3

A grapefruit oil was obtained by stirring 1000 g of a grapefruit oil having a Gardner color number of 14—for 2 hours at 120° C. and a ventilation volume of 0.1 vvm under light shielding.

### Comparative Example 7-4

A grapefruit oil was obtained by stirring 1000 g of a grapefruit oil having a Gardner color number of 14—for 72 hours at 37° C. and a ventilation volume of 0.1 vvm without light shielding (1000 lux).

### Comparative Example 7-5

A grapefruit oil was obtained by allowing 1000 g of a grapefruit oil having a Gardner color number of 14—to stand for 72 hours at 37° C. and a ventilation volume of 0.1 vvm under light shielding.

The grapefruit oils obtained in Examples 7-1 to 7-12 and Comparative Examples 7-1 to 7-5 and the untreated grapefruit oil were measured in terms of the Gardner color number in the same manner as in Test Example 1. In addition, sensory evaluation was performed in the same manner as in Test Example 1. The results are shown in Tables 8 and 9.

TABLE 8

| | | | | | | | Sensory evaluation | | |
|---|---|---|---|---|---|---|---|---|---|
| | Light | Ventilation | Treatment | | | Gardner | Evaluation | | |
| | shielding | volume | temperature | Treatment | Stirring | color | score | | Comprehensive |
| | (yes/no) | (vvm) | (° C.) | time (hr) | (yes/no) | number | (average) | Comment | evaluation |
| Untreated | — | — | — | — | — | 14− | — | — | — |
| product | | | | | | | | | |
| Example 7-1 | Yes | 0.01 | 37 | 72 | Yes | 9+ | 4.9 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Example 7-2 | Yes | 0.1 | 37 | 72 | Yes | 9 | 4.9 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Example 7-3 | Yes | 1 | 37 | 72 | Yes | 9 | 4.8 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Example 7-4 | Yes | 2 | 37 | 72 | Yes | 9+ | 4.7 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Example 7-5 | Yes | 5 | 37 | 72 | Yes | 9+ | 4.7 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Example 7-6 | Yes | 10 | 37 | 72 | Yes | 9 | 4.6 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Example 7-7 | Yes | 0.1 | 37 | 144 | Yes | 5 | 4.6 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Example 7-8 | Yes | 0.1 | 50 | 24 | Yes | 9 | 4.9 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Example 7-9 | Yes | 0.1 | 25 | 240 | Yes | 9 | 4.8 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |

TABLE 9

| | | | | | | | Sensory evaluation | | |
|---|---|---|---|---|---|---|---|---|---|
| | Light | Ventilation | Treatment | | | Gardner | Evaluation | | |
| | shielding | volume | temperature | Treatment | Stirring | color | score | | Comprehensive |
| | (yes/no) | (vvm) | (° C.) | time (hr) | (yes/no) | number | (average) | Comment | evaluation |
| Example 7-10 | Yes | 0.1 | 10 | 720 | Yes | 9 | 4.9 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Example 7-11 | Yes | 0.1 | 75 | 10 | Yes | 9 | 4.6 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Example 7-12 | Yes | 0.1 | 100 | 5 | Yes | 9 | 4.5 | Equivalent | No flavor |
| | | | | | | | | to control, | change, can |
| | | | | | | | | cannot | decolorize |
| | | | | | | | | distinguish | efficiently and |
| | | | | | | | | sensorilly | significantly |
| Comparative | Yes | Replaced | 37 | 72 | Yes | 14− | — | Not decolorized, | Cannot |
| Example 7-1 | | with nitrogen | | | | | | and thus no | decolorize |
| | | gas, sealed | | | | | | sensory | |
| | | | | | | | | evaluation | |
| Comparative | Yes | 15 | 37 | 72 | Yes | 9− | 1.9 | Aroma balance | Although can |
| Example 7-2 | | | | | | | | destroyed, not | decolorize |
| | | | | | | | | equivalent to | efficiently and |
| | | | | | | | | control, can | significantly, |
| | | | | | | | | distinguish | flavor change |
| | | | | | | | | sensorilly | was large |
| Comparative | Yes | 0.1 | 120 | 2 | Yes | 9 | 1.7 | Feel odor, not | Although can |
| Example 7-3 | | | | | | | | equivalent to | decolorize |
| | | | | | | | | control, can | efficiently and |
| | | | | | | | | distinguish | significantly, |
| | | | | | | | | sensorilly | flavor change |
| | | | | | | | | | was large |
| Comparative | No | 0.1 | 37 | 72 | Yes | 9− | 1.8 | Feel odor, not | Although can |
| Example 7-4 | (1000 lux) | | | | | | | equivalent to | decolorize |
| | | | | | | | | control, can | efficiently and |
| | | | | | | | | distinguish | significantly, |
| | | | | | | | | sensorilly | flavor change |
| | | | | | | | | | was large |
| Comparative | Yes | 0.1 | 37 | 720 | No (stand) | 13 | — | Poor | Cannot |
| Example 7-5 | | | | | | | | decolorization | decolorize |
| | | | | | | | | efficiency, | efficiently and |
| | | | | | | | | almost not | significantly |
| | | | | | | | | decolorize, | |
| | | | | | | | | and thus no | |
| | | | | | | | | sensory | |
| | | | | | | | | evaluation | |

From the results of Tables 8 and 9, it was found that the grapefruit oils were decolorized in Examples 7-1 to 7-12 as compared with the untreated product. In addition, in the sensory evaluation, Examples 7-1 to 7-12 were equivalent to the untreated product.

### Test Example 8

### Example 8-1

A five-fold concentrated product of a grapefruit oil was obtained by stirring 1000 g of a five-fold concentrated product of a grapefruit oil having a Gardner color number of 16—for 64 hours at 50° C. and a ventilation volume of 0.1 vvm under light shielding.

Air was used for the ventilation, and the ventilation and the stirring treatment were performed at the same time.

The five-fold concentrated product of the grapefruit oil obtained in Example 8-1 and the untreated five-fold concentrated product of the grapefruit oil were measured in terms of the Gardner color number in the same manner as in Test Example 1. In addition, sensory evaluation was performed in the same manner as in Test Example 1. The results are shown in Table 10.

TABLE 10

| | | | | | | | Sensory evaluation | | |
|---|---|---|---|---|---|---|---|---|---|
| | Light | Ventilation | Treatment | | | Gardner | Evaluation | | |
| | shielding | volume | temperature | Treatment | Stirring | color | score | | Comprehensive |
| | (yes/no) | (vvm) | (° C.) | time (hr) | (yes/no) | number | (average) | Comment | evaluation |
| Untreated | — | — | — | — | — | 16− | — | — | — |
| product | | | | | | | | | |
| Example 8-1 | Yes | 0.1 | 50 | 64 | Yes | 10− | 4.8 | Equivalent | No flavor |
| | | | | | | | | to control, | change, can |
| | | | | | | | | cannot | decolorize |
| | | | | | | | | distinguish | efficiently and |
| | | | | | | | | | significantly |

From the results in Table 10, it was found that the grapefruit oil was decolorized in Example 8-1 as compared with the untreated product. In addition, in the sensory evaluation, Example 8-1 was equivalent to the untreated product.

Although the present invention has been described in detail with reference to specific embodiments, it will be apparent to those skilled in the art that various modifications and variations are possible without departing from the spirit and scope of the present invention. The present application is based on Japanese Patent Application No, 2020-052999 filed on Mar. 24, 2020, contents of which are incorporated herein by reference.

1. A method for decolorizing an essential oil, the method comprising an oxidation step of oxidizing an essential oil,
wherein the oxidation step includes a step of adding water and a phenol oxidase to the essential oil, and
an addition amount of the water is 0.1 parts by mass to 1000 parts by mass with respect to 100 parts by mass of the essential oil.

2. The method for decolorizing the essential oil according to claim 1, wherein the phenol oxidase is at least one selected from the group consisting of laccase, peroxidase, and tyrosinase.

3. The method for decolorizing the essential oil according to claim 1, wherein the phenol oxidase is laccase.

4. A method for decolorizing an essential oil, the method comprising an oxidation step of oxidizing an essential oil,
wherein the oxidation step includes a ventilation step of causing air or an oxygen gas to contact the essential oil under light shielding.

5. The method for decolorizing the essential oil according to claim 4, wherein the ventilation step is performed under a condition of 100° C. or lower.

6. The method for decolorizing the essential oil according to claim 4, wherein a ventilation volume of the air or the oxygen gas in the ventilation step is 0.01 L/min to 10 L/min per 1 L of the essential oil.

7. The method for decolorizing the essential oil according to claim 4, further comprising a stirring step of stirring the essential oil.

8. A decolorized oil obtained by the method for decolorizing the essential oil according to claim 1.

9. A flavor or fragrance composition comprising the decolorized oil according to claim 8.

10. A food or drink, an oral care product, or a fragrance or cosmetic comprising the flavor or fragrance composition according to claim 9.

11. A decolorized oil obtained by the method for decolorizing the essential oil according to claim 4.

12. A flavor or fragrance composition comprising the decolorized oil according to claim 11.

13. A food or drink, an oral care product, or a fragrance or cosmetic comprising the flavor or fragrance composition according to claim 12.
